(19) United States
(12) Patent Application Publication
(10) Pub. No.: US 2007/0037825 A1
(43) Pub. Date: Feb. 15, 2007

(54) Enhancing the effect of radioimmunotherapy in the treatment of tumors

(76) Inventors: Janina Baranowska-Kortylewicz, Omaha, NE (US); Takashi Kurizaki, Kumamoto (JP); Michio Abe, Omaha, NE (US); Arne Ostman, Uppsala (SE); Kristian Pietras, Uppsala (SE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/521,299

(22) PCT Filed: Jul. 17, 2003
(86) PCT No.: PCT/IB03/03257

§ 371 Date: Feb. 14, 2006

Related U.S. Application Data
(60) Provisional application No. 60/397,347, filed on Jul. 19, 2002.

(51) Int. Cl. A61K 31/506

(52) U.S. Cl. 514/252.18

(57) ABSTRACT

4-(4-methylpiperazin-1-ylmethyl)-N-[4-methyl-3-(4-pyridin-3-yl)pyrimidin-2-ylamino)phenyl]-benzamide of the formula I 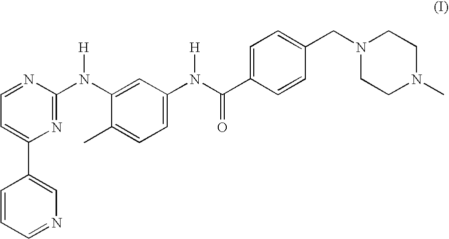 or a pharmaceutically acceptable salt thereof can be used for enhancing the effect of radioimmunotherapy of tumors.

The invention relates to the use of 4-(4-methylpiperazin-1-ylmethyl)-N-[4-methyl-3-(4-pyridin-3-yl)pyrimidin-2-ylamino)phenyl]-benzamide (hereinafter: “COMPOUND I”) or a pharmaceutically acceptable salt thereof for the manufacture of pharmaceutical compositions for enhancing the effect of radioimmunotherapy of tumors, to the use of COMPOUND I or a pharmaceutically acceptable salt thereof for treating tumors in patients subject to radioimmunotherapy, to a combination comprising COMPOUND I and a radioimmunoconjugate, and to a method of treating warm-blooded animals including humans suffering from tumors and who will be, are or were subject to radioimmunotherapy, by administering to a said animal in need of such treatment, a dose of COMPOUND I or a pharmaceutically acceptable salt thereof enhancing the effect of radioimmunotherapy.

The goal of radioimmunotherapy is to deliver ionizing radiation selectively to tumors while minimizing radiation absorbed dose to normal tissues. In creating the optimal radioimmunotherapeutic regimen, several components of the treatment are considered, including the choice of antigen, antibody, and radionuclide. The ideal antigen should be unique to the targeted tumor and not modulate or shed from the cell surface. The most effective antibodies are specific for the target antigen, have a high degree of binding affinity, clear quickly from the blood, and are not immunogenic.

The ideal characteristics of a radionuclide used for therapeutic applications include radiation emissions of a type and energy level such that the path length (X₉₀) in tissue results in optimal local energy deposition within tumors and minimal dose to distant organs. Conventional radioimmunotherapy (RIT), regardless of the radioisotope and dosing schedule fails in solid tumors. One readily identifiable cause is inadequate uptake of radioimmunoconjugates in tumor. Tumor uptake of as little as 0.01% of the injected dose, independent of the antigen status, is commonly observed in clinical studies indicating that the preponderance of radioimmunoconjugate fails to penetrate the tumor site. Total deposited radiation doses In most solid tumors are insufficient for therapy while the circulating radioisotope irradiates normal tissues. The failure of RIT in treating solid tumors is in part related to physiology of the tumor. Systemically administered monoclonal antibodies (mAbs) tend to accumulate in the periphery of the tumor and in perivascular zones. In order to reach all clonogenic tumor cells, MAbs must cross the tumor endothelium, its underlying basement membrane, the tumor stroma and parenchyma. As a result, even though tumor vessels are abnormally leaky to macromolecules, the penetration of mAbs into the tumor mass is inefficient. Usually, uptake of radiolabeled mAb is observed along capillaries at the periphery of tumor while the core of the tumor remains unlabeled. Several interrelated causes were identified at the heart of these problems and many strategies have been investigated to improve radiation doses to tumor and to limit the dose to normal tissues.

Conventional radioimmunotherapy (RIT), regardless of the radioisotope and dosing schedule uniformly fails in solid tumors because the doses delivered to the tumor are insufficient and further increases in administered doses result in radiation toxicity to normal organs.

The instant invention is a response to the need for an improved effect of radioimmunotherapy in the treatment of tumors, especially solid tumors such as colorectal and pancreatic adenocarcinomas.

It has now surprisingly been demonstrated that solid tumors can be successfully treated by radioimmunotherapy if COMPOUND I, or a pharmaceutically acceptable salt thereof, is administered during, before and/or after the radioimmunotherapy treatment period.

The present invention thus concerns the use of 4-(4-methylpiperazin-1-ylmethyl)-N-[4-methyl-3-(4-pyridin-3-yl)pyrimidin-2-ylamino)pheny]-benzamide having the formula I 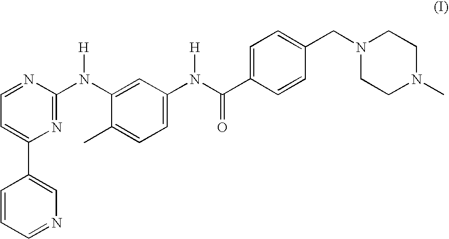
or a pharmaceutically acceptable salt thereof, for the manufacture of a medicament for enhancing the effect of radioimmunotherapy of tumors.

The preparation of COMPOUND I and the use thereof, especially as an anti-tumor agent, are described in Example 21 of European patent application EP-A-0 564 409, which was published on 6 Oct. 1993, and in equivalent applications and patents in numerous other countries, e.g. in U.S. Pat. No. 5,521,184 and in Japanese patent 2706682.

Pharmaceutically acceptable salts of COMPOUND I are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid.

The monomethanesulfonic acid addition salt of COMPOUND I (hereinafter “COMPOUND I mesylate” or “imatinib mesylate”) and a preferred crystal form thereof, e.g. the β-crystal form, are described in PCT patent application WO99/03854 published on Jan. 28, 1999. Possible pharmaceutical preparations, containing an effective amount of COMPOUND I are also described in WO99/03854 and are well known in the prior art.

4-(4-methylpiperazin-1-ylmethyl)-N-[4-methyl-3-(4-pyridin-3-yl)pyrimidin-2-ylamino)pheny]-benzamide or a pharmaceutically acceptable salt or β-crystal form thereof, will be referred herein as COMPOUND I (also known as “Imatinib” [International Non-proprietary Name]).

The present invention most particularly concerns the use of COMPOUND I or a pharmaceutically acceptable salt thereof, e.g. COMPOUND I mesylate, for the manufacture of a medicament for enhancing the effect of radioimmunotherapy in solid tumors such as pancreatic tumors; melanomas; lung cancer, e.g. small cell lung cancer; breast cancer; epidermoid carcinomas; renal-cell carcinomas; neuroendocrine tumors; genitourinary cancer, e.g. cervical, uterine, ovarian, prostate or bladder cancer; gastrointestinal cancer, e.g. gastric, colorectal adenocarcinoma or colon cancer; pancreas cancer (pancreatic adenocarcinoma); glioblastomas; head and/or neck cancer; soft-tissue sarcomas, and skin cancer, including melanoma and Kaposi's sarcoma.

In a further aspect, this invention concerns a combination, such as a combined preparation or a pharmaceutical composition, which comprises (a) N-{5-[4-(4-methyl-piperazino-methyl)-benzolamido]-2-methylphenyl}-4-(3-pyridyl)-2-pyrimidine-amine or a pharmaceutically acceptable salt thereof, e.g. the mesylate salt, and at least one compound selected from (b) a radioimmunoconjugate agent in which the active ingredients are present independently of each other in free form or in the form of a pharmaceutically acceptable salt and optionally at least one pharmaceutically acceptable carrier; for simultaneous, separate or sequential use. Such a combination will be referred hereinafter as COMBINATION OF THE INVENTION. The combinations of the present invention significantly arrest tumor growth.

In another embodiment, the instant invention provides a method of treating a warm-blooded animal, especially a human, having a tumor, comprising administering to the animal a combination, such as a combined preparation or a pharmaceutical composition, which comprises (a) N-{5-[4-(4-methyl-piperazino-methyl)-benzoylamido]-2-methylpheny}-4-(3-pyridyl)-2-pyrimidine-amine or a pharmaceutically acceptable salt thereof, e.g. COMPOUND I mesylate, and at least one compound selected from (b) a radioimmunoconjugate agent in which the active ingredients are present independently of each other in free form or in the form of a pharmaceutically acceptable salt and optionally at least one pharmaceutically acceptable carrier. Preferably the active ingredients are present in a quantity, which is jointly therapeutically effective against tumors.

The term “a combined preparation”, as used herein defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of the combination partner (a) to the combination partner (b) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a synergism, e.g. a more than additive effect, additional advantageous effects, less side effects, a combined therapeutical effect in a non-effective dosage of one or both of the combination partners (a) and (b), and very preferably a strong synergism of the combination partners (a) and (b).

COMPOUND I or a pharmaceutically acceptable salt thereof, e.g. COMPOUND I mesylate, can be administered prior, simultaneously or subsequently to the radioimmunotherapy treatment. The administration time period before or after the radioimmunotherapy treatment is preferably less than 2 months. Preferably, COMPOUND I or a pharmaceutically acceptable salt thereof, e.g. COMPOUND I mesylate, is administered within a time period of 12 days before radioimmunotherapy to 12 days after radioimmunotherapy, or 2 days before to 2 days after radioimmunotherapy.

A pharmaceutically effective amount of COMPOUND I is preferably administered between 12 hours before and 6 hours after the radioimmunotherapy treatment. In a further preferred aspect, a dose of COMPOUND I is administered less than 12 hours before and/or less than 6 hours after the radiation, preferably less than 12 hours before and/or immediately after the radiation.

In a further aspect this invention concerns, a kit for radioimmunotherapy, comprising a molecule with a radioisotope binding site linked to or on an antigen-binding fragment of an antibody or other ligand (radioimmunoconjugate) which specifically binds to a tumor-associated antigen and the COMPOUND I or a pharmaceutically acceptable salt thereof, e.g. COMPOUND I mesylate, together with instructions for their use in the treatment of tumors.

The term “radioimmunoconjugate” as used herein means antibodies, e.g. monoclonal antibodies, and other ligands, which can be attached to radioisotopes or radionuclides, e.g. by conjugation (for non-metal isotopes) or by chelation (for metal isotopes), and targeting a moiety, e.g. a tumor-associated antigen, that result in the accumulation of the radioimmunoconjugate, preferentially in tumors.

Radioimmunoconjugate as used herein includes, but is not limited to monoclonal antibodies which are selective for the cancer target cells or tissues and are linked to radionuclides. The radionuclides comprise beta, e.g. iodine-131 (¹³¹I), ⁹⁰yttrium (⁹⁰Y) or alpha, e.g. ²³¹bismuth, ²¹¹astatine, emitters. Monoclonal antibodies of the invention can be selected from a variety of targets, e.g. tenascin (an extra-cellular-matrix protein over-expressed in many tumors), CEA (carcinoembryonic antigen), TAG72 (an oncofetal antigen tumor-associated glycoprotein-72) and MUC1 (an aberrantly glycosylated epithelial mucin) epitopes. Preferably the anti-tenascin antibody is 81C6 (Reardon et al., J. Clin. Oncol. (2002) 20:1389:97), the anti-CEA antibodies are selected from the group comprising MN-14, F6 and A5B7 (Behr et al., Cancer (2002) 94:1373-81; Goldenberg J. Nucl. Med. (2002)43: 693-713), the anti-MUC1 antibodies are selected from the group comprising HMFG1 and BrE3 (Goldenberg J. Nucl. Med. (2002) 43: 693-713; Epenetos et al., J. Gynecol. Cancer. (2000) 10:44-46). The anti-TAG72 antibodies are selected from the group comprising CC49 and B72.3. The radioimmunoconjugates according to the invention are selected from the group comprising 81C6-, MN-, 14-, F6-, A5B7-, HMFG1-, BrE3-, CC49- and B72.3-nuclides, e.g. ¹³¹I-81C6, ¹³¹I-MN, ¹³¹I-14, ¹³¹I—F6, ¹³¹I-A5B7, ¹³¹I—HMFG1, ¹³¹I—BrE3, ¹³¹I—CC49, ¹³¹I—B72.3, ⁹⁰Y-81C6, ⁹⁰Y-MN, ⁹⁰Y-14, ⁹⁰Y—F6, ⁹⁰Y-A5B7, ⁹⁰Y—HMFG1, ⁹⁰Y—BrE3, ⁹⁰Y—CC49, ⁹⁰Y-B72.3. Preferably, the radioimmunoconjugates are iodine-131 (¹³¹I) labeled monoclonal antibody CC49 (¹³¹I—CC49) (Murray J L et al. Cancer (1994) 73:1057-66), ⁹⁰Y-labeled B72.3 (⁹⁰Yttrium), ¹³¹I—B72.3 (Thor A et al. J Natl. Cancer Inst. (1986) 76:995-1006), most preferably ¹³¹I—CC49, ¹³¹I—B72.3. The radioimmunoconjugates can be selected from the group comprising Tositumomab (¹³¹I-labeled form) radiolabeled anti-CD20 monoclonal antibody (CAS Registry Numbers: 208921-02-2 and 192391-48-3; U.S. Pat. No. 5,595,721), Rituximab (⁹⁰Y-labeled form) (CAS Registry Number: 174722-31-7; U.S. Pat. No. 5,763,137), Ibritumomab Tiuxetan (yttrium-90 (⁹⁰Y)-Labeled form; Zevalin®) (CAS Registry Number: 206181-63-7; U.S. Pat. No. 5,736,137), Gemtuzumab Ozogamicin (radiolabeled form) (CAS Registry Number: 220578-59-6; U.S. Pat. No. 5,773,001), Alemtuzumab or Campath-1H (radiolabeled form) (U.S. Pat. No. 5,846,534), ¹³¹I-labeled anti-CD45 antibody and ¹³¹I-labeled anti-CD33 antibody (e.g.HuM-195) (Eric L. Sievers; Cancer Chemotherapy and Pharmacology, Abstract (2000) 47:S18-S22). The radioimmunoconjugate's therapeutic dosage is well known in the art. The therapeutic dose of ⁹⁰Y-Zevalin is around 0.4 mCi/kg (15 MBq/kg) up to a maximum dose of 32 mCi (1.2 GBq).

By “solid tumors or tumors” are meant tumors and/or metastasis (wherever located) such as gliomas, pancreatic tumors; lung cancer, e.g. small cell lung cancer, breast cancer; epidermoid carcinomas; neuroendocrine tumors; gynaecological and urological cancer, e.g. cervical, uterine, ovarian, prostate, renal-cell carcinomas, testicular germ cell tumors or cancer; pancreas cancer (pancreatic adenocarcinoma); glioblastomas; head and/or neck cancer; CNS (central nervous system) cancer; bones tumors; solid pediatric tumors; haematological malignancies; AIDS-related cancer; soft-tissue sarcomas, and skin cancer, including melanoma and Kaposi's sarcoma.

The term “treatment” as used herein means curative treatment of tumors (tumor growth, metastasis, progression or invasion).

The term “curative” as used herein means efficacy in causing delay of progression, regression, more preferably even the partial or complete disappearance of tumors. The term “delay of progression” as used herein means administration of the active compound to patients being in a pre-stage or in an early phase of the disease to be treated, in which patients for example a pre-form of the corresponding disease is diagnosed or which patients are in a condition, e.g. during a medical treatment or a condition resulting from an accident, under which it is likely that a corresponding disease will develop.

By the term “quantity which is jointly therapeutically effective” there is preferably meant any quantity of the components of the combinations that, in the combination, is diminishing proliferation of cells responsible for any of the mentioned proliferative diseases (e.g. diminished tumor growth) or, preferably, even causing regression, more preferably even the partial or complete disappearance, of such cells (e.g. tumor regression, preferably cure).

Depending on species, age, individual condition, mode of administration, and the clinical picture in question, effective doses of COMPOUND I or a pharmaceutically acceptable salt thereof, e.g. COMPOUND I mesylate, is administered to warmblooded animals of about 70 kg bodyweight, for example at a dose corresponding to about 10-1000 mg of COMPOUND I free base, preferably 100-800 mg, most preferably 200 to 600 mg. For patients with an inadequate response after an assessment of response to therapy with the selected daily dosage, dose escalation can be safely considered and patients may be treated as long as they benefit from treatment and in the absence of limiting toxicities. Preferably two separate doses of COMPOUND I are given to the patient the day of radiation, e.g. one is administered a few hours before and the other just after the radioimmunotherapy treatment.

The invention relates also to a method of treating a human suffering from tumors, and who will be, is or was subject to radioimmunotherapy, which comprises administering a pharmaceutically effective amount of COMPOUND I or a pharmaceutically acceptable salt thereof to said human subject for enhancing the effect of radioimmunotherapy.

COMPOUND I is preferably administered once daily. Preferably COMPOUND I is administered within a time period from 12 days, most preferably 2 days, before the radioimmunotherapy treatment to 12 days, most preferably 2 days, after the radioimmunotherapy treatment. A pharmaceutically effective amount of COMPOUND I is preferably administered within a time period of 12 hours before to 6 hours after the radioimmunotherapy treatment. The invention relates especially to such method wherein a daily dose of 10 to 1000 mg, especially 100-800 mg, of COMPOUND I is administered. It can be shown by established test models that the COMPOUND I or a pharmaceutically acceptable salt thereof, results in the enhancement of the effect of radioimmunotherapy of tumors. Furthermore, COMPOUND I or a pharmaceutically acceptable salt thereof, results in beneficial effects in different aspect of radioimmunotherapy such as less side effects e.g. less radiation toxicity to normal organs. COMPOUND I or a pharmaceutically acceptable salt thereof shows an unexpected high potency to improve anti-tumor effects of radioimmunotherapy by an unexpected synergistic effect.

The person skilled in the pertinent art is fully enabled to select a relevant test model to prove the hereinbefore and hereinafter indicated therapeutic indications and beneficial effects (i.e. good therapeutic margin, reduction of the side effects and other advantages mentioned herein). The following Example illustrates the invention described above, but is not, however, intended to limit the scope of the invention in any way.

### EXAMPLE 1

### Enhancement of the Effect of Radioimmunotherapy of Tumors by COMPOUND I or a Pharmaceutically Acceptable Salt thereof.

Materials and Methods

Radiolabeling: Monoclonal antibodies (MAbs) are radiolabeled with [¹²⁵I]iodine or [¹³¹I]iodine for the biodistribution and/or therapy studies using the iodogen method (Fraker P J, Speck J C; Biochem Biophys Res Commun. 1978; 80:849-57). Briefly, 0.1 mg of desired protein is mixed with 1.0 mCi of Na¹²⁵I or Na¹³¹I diluted 4-fold with 0.5 M sodium phosphate buffer, pH 7.2 in a glass tube coated with 0.04 mg of iodogen. The mixture is incubated at room temperature for 30 min at which time the reaction is stopped by the addition of 0.01 mg sodium metabisulfite in 0.05 ml water. The progress of the reaction is monitored on instant thin layer chromatography strips (ITLC) using 1:4 methanol/water (v/v) as the eluant. For radiolabeling larger quantities of protein used in RIT, molar ratios of MAb, radioiodine, iodogen and metabisulfite are maintained. The entire reaction mixture is then be loaded onto a pre-equilibrated Sephadex G-10 column and the column is eluted with phosphate buffered saline. Fractions containing the desired protein are combined and tested for radiochemical purity and integrity. If the protein is used over a period of time, the amount of free iodine is determined using ITLC and/or TCA precipitation prior to use. The integrity and immunoreactivity of each preparation is analyzed using HPLC, SDS-PAGE and direct binding assays (detailed below). Specific activities of approximately 7-10 mCi per mg of protein are routinely achieved. Quality Control Analysis of Radiolabeled Antibodies: Standard operating procedures are established for the majority of the quality control procedures in our laboratories. The release criteria for radiolabeled antibodies are also established for analysis of free radioisotope, degree of protein aggregation, and immunoreactivity.

Instant Thin Layer Chromatography (ITLC): purified radiolabeled antibodies are tested for the presence of unbound iodine by thin layer chromatography. Silica-impregnated paper strips (Gelman, 1×10 cm) are used. The protein is eluted with 1:4 methanol/water mixture (v/v) in an ascending fashion. The strip is briefly air-dried and analyzed using Vista100 radioactivity scanner. The radiolabeled immunoconjugates are also analyzed by the size-exclusion HPLC (see below), and tested for radiochemical purity. Only preparations with less than 5% free radioisotope are used for animal studies.

High Performance Liquid Chromatograghy: the integrity of the radiolabeled antibody is examined using high performance liquid chromatography (HPLC). The analyses are performed using Spherogel-TSK G2000SW column (0.75×30 cm) in tandem with Spherogel-TSK G3000SW (0.75×30 cm) column, equilibrated in 67 mM sodium phosphate containing 100 mM KCl, pH 6.8 at a flow rate of 0.5 ml/min. There is a dual detection system: UV absorbance at 280 nm and radioactivity. Fractions are collected at 0.5 min intervals and the radioactive content is measured in a gamma scintillation counter. Radiolabeled preparations with less than 5% aggregates will be used for further studies.

Solid Phase Radioimmunoassays: The immunoreactivity of each of radiolabeled B72.3 and CC49 is assessed using either a solid phase 96-well based radioimmunoassay employing bovine submaxillary mucin (which exhibits the epitope seen these MAbs on the TAG-72 antigen) and bovine serum albumin as a TAG-72 negative controls or a Reacti-Gel HW-65F (Pierce, Rockford, Ill.) based assay. In the 96-well based radioimmunoassay, 50 ng of the purified proteins are added to each well of 96-well microtiter polyvinyl plates and allowed to dry. Plates are treated with 0.1 ml of 5% BSA in PBS for 1 hr at 37° C. in order to minimize non-specific protein absorption. BSA is removed and plates are stored at −20° C. until use. Before each assay plates are washed with 1% BSA in PBS and varying amounts of radiolabeled MAb (8×10⁶ cpm/ml and seven 1:2 dilutions) added in 50 μl of 1% BSA in PBS (in duplicate) to wells containing either the TAG-72-positive or the TAG-72-negative extracts. Following an overnight incubation at 4° C., the unbound immunoglobulin is removed by washing the plates with 1% BSA in PBS. The bound radioactivity is detected by cutting individual wells from the plate and measuring the radioactivity in a ã-scintillation counter. In a bead-binding assay, BSM or TAG-72 are attached to a solid-phase matrix (Reacti-Gel HW-65F) and stored at 1% BSA with 0.02% sodium azide. Coated beads are centrifuged at 500×g for 5 min, washed with 1% BSA, 0.1% Tween 20 in PBS and resuspended at 0.5 ml of binding buffer (1% BSA in PBS). Radiolabeled samples are added to each tube and vortexed every 10 min to assure complete suspension. After 1 h incubation at room temperature, the unbound radiolabeled protein is removed by repeated centrifugation and washing (3×) and the pellet counted in a gamma scintillation counter. Percent of immunoreactive MAbs is calculated as a ratio of (average cpm bound minus background) to (average cpm added minus background). The binding of radioiodinated B72.3 and CC49 to BSM bound to the matrix is generally greater than 90%. Radiolabeled MAb with significantly lower immuno-reactivity is re-tested and radiolabeling repeated, if appropriate.

SDS-Polyacrylamide Gel Electrophoresis: Radiolabeled MAb and constructs are analyzed using discontinuous SDS-PAGE. Samples are submitted to electrophoresis under non-reducing and under reducing conditions (0.5% β-mercaptoethanol, 3 min at 95° C.) using a gradient gel of 5-20% acrylamide with a stacking gel of 3% acrylamide. The radiolabeled antibody is visualized using a Molecular Dynamics phosphoimager or by autoradiography using XAR X-Ray film (Kodak, Rochester, N.Y.) with Lightning-Plus intensifying screens (DuPont, Wilmington, Del.). X-ray films are exposed at −70° C. for 1 to 7 days.

Biodistribution and Radiotherapy Studies: For most biodistribution studies ¹²⁵I-B72.3 and ¹²⁵I-CC49 are used. For therapy, both antibodies are labeled with iodine-¹³¹ and used at a dose of 0.25 mCi/mouse. According to the IACUC guidelines, all mice therapy studies are terminated at a fixed time point or when the size of SQ tumor is about 10% body weight, i.e., approximately <3,000 mm³. The following general relationship between tumor weight and volume holds for both tumor models: the weight of tumor equals 65% of the tumor volume calculated as a volume of the ellipsoid (Volume=4/3×π×(width/2)²×(length/2)). The human carcinoma cells are injected SQ into female athymic Swiss NIH mice (nu/nu), 5-6 weeks of age (5×10⁶ cells in 0.1-0.2 ml medium without serum). Consistently, 85-90% of mice grow tumors >100 mm³ in 14 to 28 days after implantation, depending on the cell line. Mice with tumors >200 mm³ will be used in COMPOUND I mesylate studies. For biodistribution studies, athymic Swiss NIH mice bearing SQ tumors or non-tumor mice (controls) are injected IV with 10 μCi/mouse of either ¹²⁵I—B72.3 or ¹²⁵I—CC49 in 0.2-mL PBS (IV). COMPOUND I mesylate is given orally. Six mice per data point are sacrificed and blood, tumor, and all the major organs including skin (up to 16 tissues per mouse) are collected, wet-weighed using an analytical balance and counted in a γ-scintillation counter. Some tumors are frozen and processed for macro-autoradiography to evaluate homogeneity of radiolabel distribution after various treatments. The percentage of the injected dose per gram (% ID/g) for each organ is determined, and tissue-to-blood ratios and radiolocalization indices (% ID/g in tumor divided by the % ID/g in the normal tissues) are calculated. The standard deviations (std) or standard errors of the mean (sem) for each tissue, at every time point, are determined. Typically the s.e.m values are less than 5% of the average values. If the s.e.m of the tissue distribution levels is greater than 15% of the average values, that given study is repeated. Data is analyzed using a local regression (LOESS) methods to produce non-parametric estimates of the relationships between time and specific tissue radiolocalizations. In therapy studies mice bearing SQ tumors or non-tumor mice (controls) receive an IV dose of 0.25 mCi/mouse of ¹³¹I—B72.3 (LS174T) or ¹³¹I—CC49 in 0.2 ml PBS. COMPOUND I mesylate is administered PO BID at 2 mg/mouse/day. Before termination of all therapy experiments, mice receive a bolus IV dose of 50 μCi ¹²⁵IUdR to measure proliferation fraction in tumors after various treatments. Selected tissues and tumors are harvested, counted in a γ-scintillation counter, and examined histologically. Sections of tumors with ¹²⁵IUdR are subjected to micro-autoradiography after the decay of residual ¹³¹I activity and ¹²⁵IUdR bound to DNA is determined using Wako's DNA extraction kit.

Results:

Antibodies: Two MAb are selected for RIT studies: B72.3 (Rosenblum M G et al. Clin Cancer Res 1999 5:953-61; Thor A et al. J Natl Cancer Inst 1986 76:995-1006) and CC49. B72.3 is a prototype MAb which recognizes the same antigen as CC49, a high-molecular weight glycoprotein complex designated as tumor-associated glycoprotein-72 (TAG-72). Both antibodies have a significant reactivity with over 85% of adenocarcinomas including pancreatic cancer and only a minimal reactivity with normal tissues. B72.3 is an excellent diagnostic agent but RIT clinical trials with this antibody uniformly failed. B72.3 and CC49, when labeled with therapeutic radioisotopes arrest or significantly delay growth of SQ adenocarcinomas in mice in a dose-dependent manner. The degree of the tumor response is also governed by the size of the tumor at the start of RIT, the choice of antibody and the radioisotope. For example, a single dose of 0.5 mCi ¹³¹I—CC49 produces profound tumor regression and cures when tested in SQ LS174T human colorectal adenocarcinoma xenografts in athymic mice. Sixty percent of LS174T tumors treated with 0.5 mCi of ¹³¹I—CC49 regress completely. When similar doses of ¹³¹I—B72.3 are used in the same tumor model, there is a growth delay but no cures. However, escalating doses of ¹³¹I—B72.3 produce cures and tumor growth arrest in mice. Regrettably, these results cannot be reproduced in a clinical situation and these antibodies like most other failed in clinical studies in solid tumors.

When planning the evaluation of augmented RIT, the less effective, first generation monoclonal antibody B72.3 rather than CC49 is elected to be used. The advantages of the adjuvant treatment are more apparent in a condition where the degree of response to RIT is less than optimal. Moreover, this reflects the more difficult clinical situation that is encountered in RIT of adenocarcinomas. This approach works well in the preliminary model experiments in the LS174T tumors. In SW1990 pancreatic adenocarcinoma, studies are done with ¹³¹I—CC49 at a 0.25-mCi dose.

Tumor Models: LS174T is a human colorectal adenocarcinoma model tested extensively with a variety of antibodies including B72.3 and CC49. The availability of data from various sources pertinent to the proposed studies allows rapid evaluation and comparison of treatments. SW1990 is a well to moderately well differentiated human pancreatic adenocarcinoma. There is extensive immunological cross-reaction between SW1990 pancreatic cancer mucin and LS174T colon cancer mucin. SW 1990 can be specifically targeted with ¹²⁵I—B72.3 and ¹²⁵I—CC49.

Effect of COMPOUND I Mesylate on Radiosensitivity of In Vitro Grown Cells:

Arrest of LS174T cells in the G1 phase in the presence of pharmacologically relevant concentrations of COMPOUND I mesylate prompted the investigation of the combined effects of radiation and COMPOUND I mesylate. Cells are grown as a monolayer and treated with various concentrations of COMPOUND I mesylate followed by irradiation at 1.95 Gy/min for total doses of 1 Gy and 6 Gy. Neither of these two cells lines had any particularly unusual sensitivity to radiation. As expected, the 6 Gy dose produced about 60% cell kill whereas a sublethal dose of 1 Gy retarded the cell growth by less than 2%. On the basis of these results, it is apparent that even though COMPOUND I mesylate has an effect on the cell cycle, this effect is not sufficient in vitro to synchronize all cells and render the entire population less (LS174T) radiosensitive. The effect of combined treatment with ¹³¹I-labeled antibodies and COMPOUND I mesylate in vitro is also tested. Two monoclonal antibodies are used: ¹³¹I-anti-CEA (LS174T expresses CEA) and ¹³¹I—B72.3. In either case neither additive nor synergistic effects are measurable.

Effect of COMPOUND I on Radiosensitivity of Adenocarcinoma Xenografts:

Potential deterioration of tumor radiosensitivity related to the COMPOUND I mesylate-induced G1 arrest of LS174T cells is also investigated in SQ xenografts in athymic nude mice. But there are no statistical differences between radiation plus COMPOUND I mesylate-treated mice and radiation only: P=0.127 (all P values obtained in a Mantel-Cox 20 logrank analysis).

Potentiation of Radioimmunotherapy with COMPOUND I Mesylate:

COMPOUND I mesylate-enhanced cancer radioimmunotherapy trials are conducted in mice to determine the in vivo mechanism by which COMPOUND I mesylate improves RIT and to determine the dosing timeline. A summary of data collected is shown in Table 1. Tumors are implanted SQ (5×10⁶ LS174T cells/mouse) and allowed to grow for 10 days. Mice are randomized into four groups: (1) no treatment (NT); (2) ¹³¹I—B72.3 only; (3) COMPOUND I mesylate only and (4) ¹³¹I—B72.3 plus COMPOUND I mesylate. Tumor size is measured every three days and tumor volumes calculated. Data is plotted as a tumor growth relative to tumor size on day 3 when the first dose of COMPOUND I mesylate is given. On the day of ¹³¹I—B72.3 administration the average tumor size is 270 mm³. One week after the 0.25-mCi dose of ¹³¹I—B72.3, tumor volumes in mice treated with a combination COMPOUND I mesylate-RIT are less that 50% of the control, i.e., untreated tumors. During this same time, RIT alone produced approximately a 10% decrease in volume. Treatment with COMPOUND I mesylate alone had no effect. The change in quadrupling time (Tq) is calculated on day 10 for the controls (termination day due to the excessive tumor burden >3,000 mm²) and on day 28 after ¹³¹IB72.3 for the rest of mice (Table 1).

TABLE 1
Effect of RIT and combination RIT/COMPOUND I mesylate on doubling
times of LS174T xenografs in athymic mice. (* day 10; **day 28)

| | T_{q} (days) | Avg(std) | Relative tumor growth |
|---|---|---|---|
| No treatment n = 6 | 7.74* | (1.34) | 1 |
| COMPOUND I mesylate n = 10 | 7.75* | (1.20) | 1 |
| ¹³¹I-B72.3 n = 6 | 18.95** | (2.98) | 2.4 |
| COMPOUND I mesylate + ¹³¹I-B72.3 n = 9 | 40.63** | (8.43) | 5.2 |

The inclusion of COMPOUND I mesylate in the ¹³¹I-B72.3 therapy protocol improves anti-tumor effects by about 220%. Tq of COMPOUND I mesylate − ¹³¹B72.3-treated mice is delayed over 5 fold compared to non-treated controls.

A similar study is conducted in SW1990. The response of tumor to the combination therapy is significantly improved compared to any treatment applied alone. After day 38, statistical differences emerged between ¹³¹I—CC49 alone and COMPOUND I mesylate+¹³¹I—CC49 groups of mice with 100-200 mm³ tumors on the day of antibody treatment (0.001<p<0.01). A significant arrest of tumor growth is apparent. In both models, tumor response to a single bolus dose of 0.25 mCi ¹³¹I—CC49 in combination with COMPOUND I mesylate is equivalent to the response obtained with a nearly two times greater dose when ¹³¹I—CC49 is used alone. Taken together, these results suggest that COMPOUND I or a pharmaceutically acceptable salt thereof, e.g. COMPOUND I mesylate, has an unexpected potential to improve the effect of radioimmunotherapy treatment.

### EXAMPLE 2

Capsules with COMPOUND I mesylate (optionally in its β-crystal form). Capsules containing 119.5 mg of COMPOUND I mesylate corresponding to 100 mg of COMPOUND I (free base) as active substance are prepared in the following composition:

| | | |
|---|---|---|
| COMPOUND I mesylate | 119.5 | mg |
| Avicel | 200 | mg |
| PVPPXL | 15 | mg |
| Aerosil | 2 | mg |
| Magnesium stearate | 1.5 | mg |
| | 338.0 | mg |

The capsules are prepared by mixing the components and filling the mixture into hard gelatin capsules, size 1.

These examples illustrate the invention without in any way limiting its scope.

1. (canceled)

2. A combination which comprises (a) N-{5-[4-(4-methyl-piperazino-methyl)-benzoylamido]-2-methylphenyl}-4-(3-pyridyl)-2-pyrimidine-amine and at least one compound selected from (b) a radioimmunoconjugate agent in which the active ingredients are present independently of each other in free form or in the form of a pharmaceutically acceptable salt and optionally at least one pharmaceutically acceptable carrier; for simultaneous, separate or sequential use.

3. A method of treating a human suffering from tumors and who will be, is or was subject to radioimmunotherapy, which comprises administering to a said human in need of such treatment, a dose of 4-(4-methylpiperazin-1-ylmethyl)-N-[4-methyl-3-(4-pyridin-3-yl)pyrimidin-2-ylamino)phenyl]-benzamide of the formula I or a pharmaceutically acceptable salt thereof, for enhancing the effect of radioimmunotherapy.

4. A combination according to claim 2 wherein a daily dose of 10 to 1000 mg of 4-(4-methylpiperazin-1-ylmethyl)-N-[4-methyl-3-(4-pyridin-3-yl)pyrimidin-2-ylamino)phenyl]-benzamide of the formula I is administered to an adult human.

5. A combination according to claim 2 wherein a pharmaceutically acceptable acid addition salt of 4-(4-methylpiperazin-1-ylmethyl)-N-[4-methyl-3-(4-pyridin-3-yl)pyrimidin-2-ylamino)phenyl]-benzamide of the formula I is administered.

6. A combination according to claim 5 wherein a monomethanesulfonate salt of 4-(4-methylpiperazin-1-ylmethyl)-N-[4-methyl-3-(4-pyridin-3-yl)pyrimidin-2-ylamino)phenyl]-benzamide of the formula I is administered.

7. A combination according to claim 2 wherein the radioimmunoconjugate is selected from the group comprising ¹³¹I-81C6, ¹³¹I-MN, ¹³¹I-14, ¹³¹I—F6, ¹³¹I-A5B7, ¹³¹I-HMFG1, ¹³¹I-BrE3, ¹³¹I—CC49, ¹³¹I—B72.3, ⁹⁰Y-81C6, ⁹⁰Y-MN, ⁹⁰Y-14, ⁹⁰Y—F6, ⁹⁰Y-A5B7, ⁹⁰Y—HMFG1, ⁹⁰Y—BrE3, ⁹⁰Y—CC49 and ⁹⁰Y—B72.3.

8. A combination according to claim 7 wherein the radioimmunoconjugate is selected from the group consisting of ¹³¹I—CC49 and ¹³¹I—B72.3.

9. A combination according to claim 2 wherein 4-(4-methylpiperazin-1-ylmethyl)-N-[4-methyl-3-(4-pyridin-3-yl)pyrimidin-2-ylamino)phenyl]-benzamide of the formula I is administered within a time period of 12 days before to 12 days after radioimmunotherapy.

10. A method of treating a warm-blooded animal, especially a human having a tumor, comprising administering to said animal a combination, such as a combined preparation or a pharmaceutical composition, which comprises (a) N-{5-[4-(4-methyl-piperazino-methyl)-benzoylamido]-2-methylphenyl}-4-(3-pyridyl)-2-pyrimidine-amine, and at least one compound selected from (b) a radioimmunoconjugate agent in which the active ingredients are present independently of each other in free form or in the form of a pharmaceutically acceptable salt and optionally at least one pharmaceutically acceptable carrier.

11. A method according to claim 10 for enhancing the effect of radioimmunotherapy in tumors selected from pancreatic tumors, lung cancer, breast cancer, epidermoid carcinomas, renal-cell carcinomas, neuroendocrine tumors, gynaecological cancer, urological cancer, gastrointestinal cancer, colorectal adenocarcinoma or colon cancer, pancreatic adenocarcinoma; glioblastomas, head and/or neck cancer, central nervous system cancer, bones tumors, solid pediatric tumors, haematological malignancies, AIDS-related cancer, soft-tissue sarcomas, and skin cancer.

12. A kit for radioimmunotherapy, comprising:
a) a radioimmunoconjugate agent which specifically binds to a tumor-associated antigen, and
b) 4-(4-methylpiperazin-1-ylmethyl)-N-[4-methyl-3-(4-pyridin-3-yl)pyrimidin-2-yl-amino)phenyl]-benzamide of the formula I or a pharmaceutically acceptable salt thereof.

13. A method according to claim 3 wherein a daily dose of 10 to 1000 mg of 4-(4-methylpiperazin-1-ylmethyl)-N-[4-methyl-3-(4-pyridin-3-yl)pyrimidin-2-ylamino)phenyl]-benzamide of the formula I is administered to an adult human.

14. A method according to claim 3 wherein a pharmaceutically acceptable acid addition salt of 4-(4-methylpiperazin-1-ylmethyl)-N-[4-methyl-3-(4-pyridin-3-yl)pyrimidin-2-yl-amino)phenyl]-benzamide of the formula I is administered.

15. A method according to claim 3 wherein a monomethanesulfonate salt of 4-(4-methylpiperazin-1-ylmethyl)-N-[4-methyl-3-(4-pyridin-3-yl)pyrimidin-2-ylamino)phenyl]-benzamide of the formula I is administered.

16. A method according to claim 3 wherein 4-(4-methylpiperazin-1-ylmethyl)-N-[4-methyl-3-(4-pyridin-3-yl)pyrimidin-2-ylamino)phenyl]-benzamide of the formula I is administered within a time period of 12 days before to 12 days after radioimmunotherapy.

17. A method according to claim 3 for enhancing the effect of radioimmunotherapy in tumors selected from pancreatic tumors, lung cancer, breast cancer, epidermoid carcinomas, renal-cell carcinomas, neuroendocrine tumors, gynaecological cancer, urological cancer, gastrointestinal cancer, colorectal adenocarcinoma or colon cancer, pancreatic adenocarcinoma; glioblastomas, head and/or neck cancer, central nervous system cancer, bones tumors, solid pediatric tumors, haematological malignancies, AIDS-related cancer, soft-tissue sarcomas, and skin cancer.
